(19) United States
(12) Patent Application Publication
(10) Pub. No.: US 2026/0294798 A1
(43) Pub. Date: Oct. 1, 2026

(54) OPHTHALMIC DEVICE FOR TREATMENT OF DRY EYE

(71) Applicant: Bausch + Lomb Ireland Limited, Dublin (IE)

(72) Inventors: Alok Kumar Awasthi, Pittsford, NY (US); Sankarsan Biswas, Rochester, NY (US); Feng-Yang Shih, Lafayette, CO (US); Jade Russell, Perry, NY (US); James Anthony DiBella, Jr., Penfield, NY (US); Anna Flach, Lafayette, CO (US); Alana Ingham, Rochester, NY (US)

(21) Appl. No.: 19/556,251

(22) Filed: Mar. 4, 2026

Related U.S. Application Data
(60) Provisional application No. 63/778,557, filed on Mar. 27, 2025.

(51) Int. Cl. A61K 9/00; A61K 31/4725; A61K 47/34; G02C 7/04

(52) U.S. Cl. CPC A61K 9/0051; A61K 31/4725; A61K 47/34; G02C 7/049

(57) ABSTRACT

An ophthalmic device is a polymerization product of a monomeric mixture including (a) a lifitegrast or a salt thereof-containing hydrophilic polymer comprising a releasable linking group and an ethylenically unsaturated reactive group, and (b) an ophthalmic device-forming comonomer.

### PRIORITY CLAIM

The present application claims priority to U.S. Provisional Patent Application Ser. No. 63/778,557, entitled “Ophthalmic Device for Treatment of Dry Eye,” filed Mar. 27, 2025, the content of which is incorporated by reference herein in its entirety.

### BACKGROUND

Soft contact lenses have been available since the 1980s. It is important that contact lenses be comfortable and safe to wear. However, while there are many people who can successfully wear contact lenses, there are a number of people who can wear contact lenses for only a short period of time due to, for example, contact lens related dry eye. Symptoms of this disorder include, for example, thin and/or unstable tear films, corneal staining and subjective symptoms such as ocular discomfort, burning/stinging and dryness. Contact lens wear may trigger the onset of these symptoms or may exacerbate the symptoms.

Although lenses with high water contents are softer, more lubricious and more comfortable to wear, such lenses may not have one or more properties useful to provide comfortable and safe wearing of the contact lenses. For example, a particular problem associated with high water content contact lenses is evaporative corneal dehydration. As free water in the lens is lost due to evaporation, it is replaced with water from the cornea. Evaporative water loss at the anterior lens surface is a potential cause of contact lens dehydration and of post-lens tear-film depletion, which in turn, may lead to discomfort, dry eye, corneal staining and/or other damage to the eye.

### SUMMARY

In accordance with an aspect of the present disclosure, an ophthalmic device comprises a polymerization product of a monomeric mixture comprising (a) a lifitegrast or a salt thereof-containing hydrophilic polymer comprising a releasable linking group and an ethylenically unsaturated reactive group, and (b) an ophthalmic device-forming comonomer.

In accordance with another aspect of the present disclosure, a method for making an ophthalmic device comprises:
(a) subjecting a monomeric mixture comprising (i) a lifitegrast or a salt thereof-containing hydrophilic polymer comprising a releasable linking group and an ethylenically unsaturated reactive group, and (ii) an ophthalmic device-forming comonomer to polymerization conditions to provide a polymerized ophthalmic device, and
(b) hydrating the polymerized ophthalmic device.

In accordance with yet another aspect of the present disclosure, a method for delivering lifitegrast or a salt thereof to an eye of a subject comprises:
(a) placing an ophthalmic device in an eye of a subject, wherein the ophthalmic device comprises a polymerization product of a monomeric mixture comprising (i) a lifitegrast or a salt thereof-containing hydrophilic polymer comprising a releasable linking group and an ethylenically unsaturated reactive group, and (ii) an ophthalmic device-forming comonomer, and
(b) delivering the lifitegrast or a salt thereof to an eye of a subject by contacting the ophthalmic device with one of light having a wavelength of about 300 nanometers (nm) to about 700 nm or with an esterases or enzymes on the eye, in a tear film or on a flora of the eye.

### DETAILED DESCRIPTION

Various illustrative embodiments described herein include an ophthalmic device comprises a polymerization product of a monomeric mixture comprising (a) a lifitegrast or a salt thereof-containing hydrophilic polymer comprising a releasable linking group and an ethylenically unsaturated reactive group, and (b) an ophthalmic device-forming comonomer for delivering lifitegrast or a salt thereof to an eye of a user.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

As used in this disclosure, the word “comprises” or “comprising” is intended as an open-ended transition meaning the inclusion of the named elements, but not necessarily excluding other unnamed elements. The phrase “consists essentially of” or “consisting essentially of” is intended to mean the exclusion of other elements of any essential significance to the composition. The phrase “consisting of” or “consists of” is intended as a transition meaning the exclusion of all but the recited elements with the exception of only minor traces of impurities.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means±20% of the stated value, ±15% of the stated value, ±10% of the stated value, +5% of the stated value, ±3% of the stated value, or +1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

Representative examples of ethylenically unsaturated reactive groups for use herein include, by way of example, a (meth)acrylate-containing reactive end group, a (meth)acrylamide-containing reactive end group, an allyl-containing reactive end group, a vinyl-containing reactive end group, a vinylcarbonate-containing reactive end group, a vinylcarbamate-containing reactive end group, a styrene-containing reactive end group, an itaconate-containing reactive end group, a vinyloxy-containing reactive end group, a fumarate-containing reactive end group, a maleimide-containing reactive end group, a vinylsulfonyl reactive end group and the like.

In a non-limiting illustrative embodiment, a (meth)acrylate-containing reactive end group can be represented by the structure:

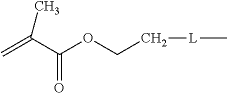

wherein L is a linking group or bond. Suitable linking groups include, for example, a heteroatom such as O, any divalent hydrocarbon radical or moiety such as independently a straight or branched, substituted or unsubstituted C₁-C₆ alkyl group, a substituted or unsubstituted C₃-C₁₂ cycloalkyl group, a substituted or unsubstituted C₄-C₁₂ cycloalkylalkyl group, a substituted or unsubstituted C₃-C₁₂ cycloalkenyl group, a substituted or unsubstituted C₆-C₁₂ aryl group, a substituted or unsubstituted C₇-C₁₂ arylalkyl group and substituted and unsubstituted ether-containing groups.

As used herein, the term “(meth)” denotes an optional methyl substituent. Thus, terms such as “(meth)acrylate” denotes either methacrylate or acrylate, and “(meth)acrylamide” denotes either methacrylamide or acrylamide.

As used herein, the term “ophthalmic device” refers to devices that reside in or on the eye. These ophthalmic devices can provide optical correction, wound care, drug delivery, diagnostic functionality or cosmetic enhancement or effect or a combination of these properties. Representative examples of such devices include, but are not limited to, soft contact lenses, e.g., a soft, hydrogel lens; soft, non-hydrogel lens and the like, hard contact lenses, e.g., a hard, gas permeable lens material and the like, intraocular lenses, overlay lenses, ocular inserts, optical inserts and the like. As is understood by one skilled in the art, a lens is considered to be “soft” if it can be folded back upon itself without breaking. A contact lens can be in a dry state or a wet state. A “dry state” refers to a soft contact lens in a state prior to hydration or the state of a hard lens under storage or use conditions. A “wet state” refers to a soft contact lens in a hydrated state.

Ophthalmic drug delivery faces significant obstacles due to anatomical and physiological barriers, such as tear drainage and limited epithelial transport, which restrict drug bioavailability to around 5% or less. These barriers impede the penetration and absorption of therapeutic agents, making effective treatment challenging. An optimal delivery system must address these issues by enhancing bioavailability and enabling controlled drug release at the target site.

Lifitegrast or salt thereof is a well-established medication for the treatment of dry eye disease. Ophthalmic lifitegrast or salt thereof is specifically used to alleviate the signs and symptoms associated with this condition. Classified as a lymphocyte function-associated antigen-1 (LFA-1) antagonist, lifitegrast or salt thereof functions by reducing inflammation in the eye tissues.

Conventional methods like eye drops and ointments have been used to deliver lifitegrast or a salt thereof. However, these delivery methods are inefficient, requiring frequent applications and often resulting in poor patient compliance and incorrect dosing. While advanced formulations such as gels, viscous solutions, and colloidal systems have been developed, they have yet to achieve satisfactory outcomes.

The non-limiting illustrative embodiments disclosed herein overcome the foregoing drawbacks by providing an ophthalmic device that can deliver lifitegrast or a salt thereof to the eye of a user in a continuous and sustained release manner. By providing significantly greater drug bioavailability to the cornea compared to the traditional methods discussed above, the ophthalmic devices such as contact lenses of the non-limiting illustrative embodiments described herein can be utilized as a controlled drug delivery platform. This can be achieved by forming the ophthalmic devices from a polymerization product of a monomeric mixture comprising (a) a lifitegrast or a salt thereof-containing hydrophilic polymer comprising a releasable linking group and an ethylenically unsaturated reactive group, and (b) an ophthalmic device-forming comonomer. The lifitegrast or a salt thereof can be delivered to an eye of a subject by contacting the ophthalmic device with one of light having a wavelength of about 300 nanometers (nm) to about 700 nm or with an esterases or enzymes on the eye, in a tear film or on a flora of the eye thereby releasing the lifitegrast or a salt thereof in a sustained release.

In non-limiting illustrative embodiments, the ophthalmic devices disclosed herein are a polymerization product of a monomeric mixture comprising (a) a lifitegrast or a salt thereof-containing hydrophilic polymer comprising a releasable linking group and an ethylenically unsaturated reactive group, and (b) an ophthalmic device-forming comonomer. In general, lifitegrast is represented by the following structure:

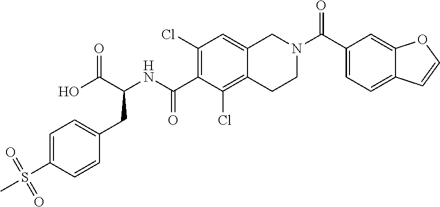 [SMILES: CS(=O)(=O)c1ccc(C[C@H](NC(=O)c2c(Cl)cc3c(c2Cl)CCN(C(=O)c2ccc4ccoc4c2)C3)C(=O)O)cc1]

and a salt of lifitegrast is represented by the following structure:

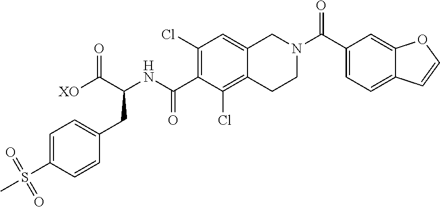

where x is a salt such as a sodium salt.

In some embodiments, a lifitegrast or a salt thereof-containing hydrophilic polymer can have one or more releasable linking groups. In some embodiments, a releasable linking group refers to a releasable linking group that includes at least one bond that can be broken under physiological conditions, e.g., where the releasable linking group is a group linking the lifitegrast or a salt thereof to a hydrophilic polymer comprising an ethylenically unsaturated reactive group via a pH-labile bond, an acid-labile bond, a base-labile bond, an oxidatively labile bond, a metabolically labile bond, a biochemically labile bond or an enzyme-labile bond, or other conditions such as a standard chemical reaction via a chemical-labile bond, e.g., a hydrolysis or substitution reaction, or via a photo-labile bond, e.g., exposure to light such as UV or near UV light, e.g., a photosensitive bond. If the releasable linking groups are naturally cleaved under physiological conditions or cellular physiological conditions, then the hydrophilic polymer is biodegradable. In some embodiments, the releasable linking groups result in a loss of the lifitegrast or a salt thereof from a side chain atom of the lifitegrast or a salt thereof-containing hydrophilic polymer thereby releasing the lifitegrast or a salt thereof.

In some embodiments, the releasable linking group includes ester groups. In the case of ester bonds, the esters undergo hydrolysis and are also catalytically cleaved by esterases thereby releasing the lifitegrast or a salt thereof from the lifitegrast or a salt thereof-containing hydrophilic polymer.

In some embodiments, the releasable linking group includes a photocleavable linking group. Suitable photocleavable linking groups include, for example, a nitrobenzyl group, phenacyl group, pyronin group, benzoin group and coumarin group. In one aspect, the photocleavable linking group has at least two reactive groups. The first reactive group allows linking of the photocleavable linking group to the hydrophilic polymer. The second reactive group allows linking of the photocleavable linking group to the lifitegrast or a salt thereof. The former handle is preferably stable and the latter handle is preferably amenable to photolysis such that the lifitegrast or a salt thereof is released from the photocleavable lifitegrast conjugate upon exposure to light of the appropriate wavelength. The photocleavable group therefore breaks its bond with the lifitegrast or a salt thereof, removing the charged groups from the lifitegrast or a salt thereof. The lifitegrast or a salt thereof is then in its native form with no additions.

In some embodiments, a lifitegrast or a salt thereof-containing hydrophilic polymer comprising a releasable linking group and an ethylenically unsaturated reactive group can include, for example, a lifitegrast or a salt thereof-containing glycosaminoglycan (GAG) polymer comprising a GAG having a polymer backbone and a first side chain comprising an ethylenically unsaturated reactive-containing residue grafted onto the polymer backbone and a second side chain comprising a lifitegrast or a salt thereof residue grafted onto the polymer backbone. In other words, the ethylenically unsaturated reactive-containing residue grafted onto the GAG will have a reactive functional group in the polymer backbone capable of being grafted onto a reactive functional group of an ethylenically unsaturated reactive monomer and a reactive functional group in the polymer backbone capable of being grafted onto a reactive functional group of lifitegrast or salt thereof. For example, a carboxylic acid group of lifitegrast or a salt thereof can react with a hydroxyl group in the polymer backbone of a GAG such as hyaluronic acid to form a lifitegrast or a salt thereof-containing hyaluronic acid polymer comprising a releasable linking group in the form of an ester group.

A GAG is one molecule with many alternating subunits. A GAG is one molecule with many alternating subunits. In general, GAGs are represented by the formula A-B-A-B-A-B, where A is uronic acid and B is an amino sugar that may or may not be either O- or N-sulfated, where the A and B units can be heterogeneous with respect to epimeric content or sulfation. Any natural or synthetic polymer containing uronic acid can be used. Other GAGs are sulfated at different sugars. There are many different types of GAGs having commonly understood structures such as, for example, chondroitin sulfate (e.g., chondroitin 4- and 6-sulfates), heparan, heparin sulfate, heparosan, dermatan, dermatan sulfate, hyaluronic acid or a salt thereof, e.g., sodium hyaluronate or potassium hyaluronate, keratan sulfate, and other disaccharides such as sucrose, lactulose, lactose, maltose, trehalose, cellobiose, mannobiose and chitobiose. GAGs can be purchased from Sigma, and many other biochemical suppliers such as HTL Biotechnology (France). In an illustrative embodiment, the GAG is hyaluronic acid. In another illustrative embodiment, the GAG is chondroitin sulfate.

In an illustrative embodiment, a GAG for use herein can have a weight average molecular weight ranging from about 10,000 to about 3,000,000 Daltons (Da) in which the lower limit is from about 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about up to 2,800,000 Da, where any of the lower limits can be combined with any of the upper limits. In an illustrative embodiment, a GAG for use herein can have a weight average molecular weight ranging from about 1,000,000 to about 3,000,000 Da.

Hyaluronic acid is a well-known, naturally occurring, water soluble biodegradable polymer composed of two alternatively linked sugars, D-glucuronic acid and N-acetylglucosamine, linked via alternating β-(1,4) and β-(1,3) glycosidic bonds. Hyaluronic acid is a non-sulfated GAG. The polymer is hydrophilic and highly viscous in aqueous solution at relatively low solute concentrations. It often occurs naturally as the sodium salt, sodium hyaluronate. Methods of preparing commercially available hyaluronan and salts thereof are well known. Hyaluronan can be purchased from Seikagaku Company, Clear Solutions Biotech, Inc., Pharmacia Inc., Sigma Inc., HTL Biotechnology, Contipro and Bloomage Biotechnology Corporation, and many other suppliers. Hyaluronic acid has repeating units of the structure represented by the following formula:

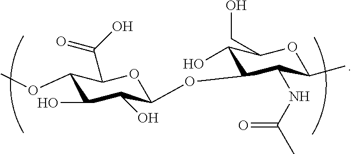

Accordingly, the repeating units in hyaluronic acid can be as follows:

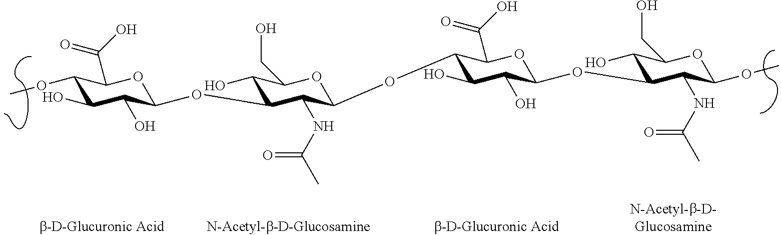

In general, hyaluronic acid or a salt thereof can have from about 2 to about 1,500,000 disaccharide units. In an embodiment, hyaluronic acid or a salt thereof can have a weight average molecular weight ranging from about 10,000 to about 3,000,000 Da in which the lower limit is from about 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about up to 2,800,000 Da, where any of the lower limits can be combined with any of the upper limits.

Chondroitin sulfate is a linear sulfated polysaccharide composed of repeating β-D-glucuronic acid (GlcA) and N-acetyl-β-D-galactosamine (GalNAc) units arranged in the sequence by GlcA-β(1,3)-GalNAc-β(1,4) glycosidic bonds. In an embodiment, chondroitin sulfate has one or more repeating units of the structure represented by the following formula:

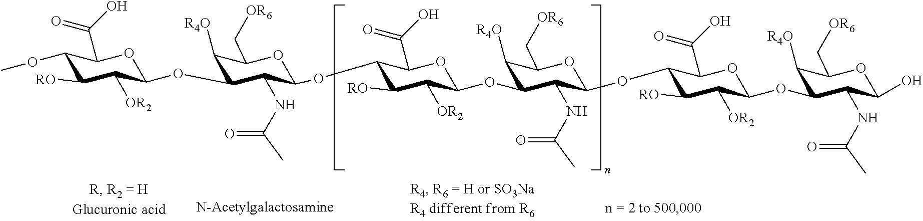

In an illustrative embodiment, chondroitin sulfate has repeating units of the structure represented by the following formula:

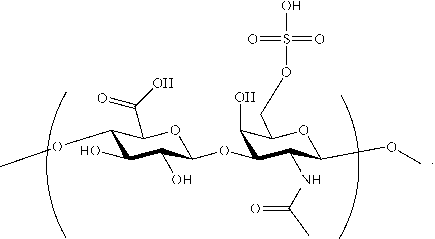

In general, chondroitin sulfate can have from about 2 to about 1,500,000 repeating units. In an embodiment, chondroitin sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about 3,000,000 Da where any of the lower limits can be combined with any of the upper limits or any of the upper limits can be combined with any of the upper limits.

In an illustrative embodiment, dermatan sulfate has repeating units of the structure represented by the following formula:

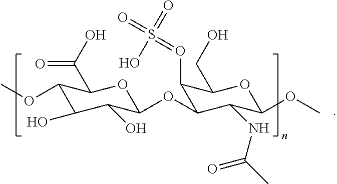

In general, dermatan sulfate can have from about 2 to about 1,500,000 repeating units. In an embodiment, dermatan sulfate can have a weight average molecular weight ranging from about 1,000 to about 2,000,000 Da in which the lower limit is from about 1,000, 5,000, 10,000, about 20,000, about 30,000, about 40,000, about 50,000, about 60,000, about 70,000, about 80,000, about 90,000, or about 100,000 Da, and the upper limit is about 200,000, about 300,000, about 400,000, about 500,000, about 600,000, about 700,000, about 800,000, about 900,000, about 1,000,000, or about 2,000,000 Da where any of the lower limits can be combined with any of the upper limits or any of the upper limits can be combined with any of the upper limits.

In an illustrative embodiment, heparin and heparin sulfate has repeating units of the structure represented by the following formula:

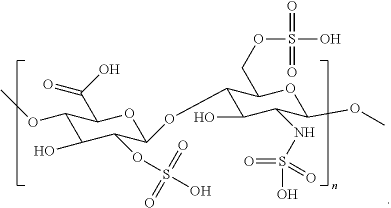

In general, heparin and heparin sulfate can have from about 2 to about 1,500,000 repeating units. In an embodiment, heparin and heparin sulfate can have a weight average molecular weight ranging from about 1,000 to about 3,000,000 Da in which the lower limit is from about 1,000, 5,000, 10,000, about 20,000, about 30,000, about 40,000, about 50,000, about 60,000, about 70,000, about 80,000, about 90,000, or about 100,000 Da, and the upper limit is about 40,000, 100,000, 200,000, about 300,000, about 400,000, about 500,000, about 600,000, about 700,000, about 800,000, about 900,000, about 1,000,000, or about 3,000,000 Da where any of the lower limits can be combined with any of the upper limits or any of the upper limits can be combined with any of the upper limits.

In an illustrative embodiment, keratan sulfate has repeating units of the structure represented by the following formula:

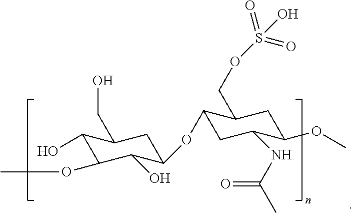

In general, keratan sulfate can have from about 2 to about 1,500,000 repeating units. In an embodiment, keratan sulfate can have a weight average molecular weight ranging from about 10,000 to about 3,000,000 Da in which the lower limit is from about 5,000, 10,000, about 20,000, about 30,000, about 40,000, about 50,000, about 60,000, about 70,000, about 80,000, about 90,000, or about 100,000 Da, and the upper limit is about 100,000, 200,000, about 300,000, about 400,000, about 500,000, about 550,000, 600,000, about 700,000, about 800,000, about 900,000, about 1,000,000, or about 3,000,000 Da where any of the lower limits can be combined with any of the upper limits or any of the upper limits can be combined with any of the upper limits.

A representative example of a lifitegrast or a salt thereof-containing GAG polymer is set forth below:

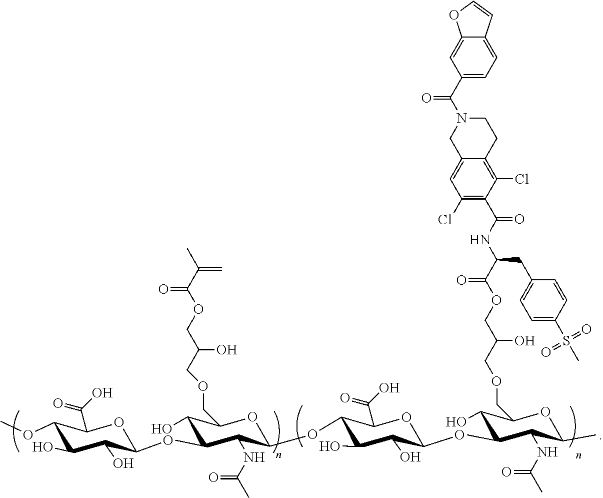

In some embodiments, a lifitegrast or a salt thereof-containing GAG polymer comprising a GAG having a polymer backbone and a first side chain comprising an ethylenically unsaturated reactive-containing residue grafted onto the polymer backbone and a second side chain comprising a lifitegrast or a salt thereof residue grafted onto the polymer backbone such as a lifitegrast or a salt thereof-containing hyaluronic acid polymer can be made by a reaction scheme seen below in Scheme I.

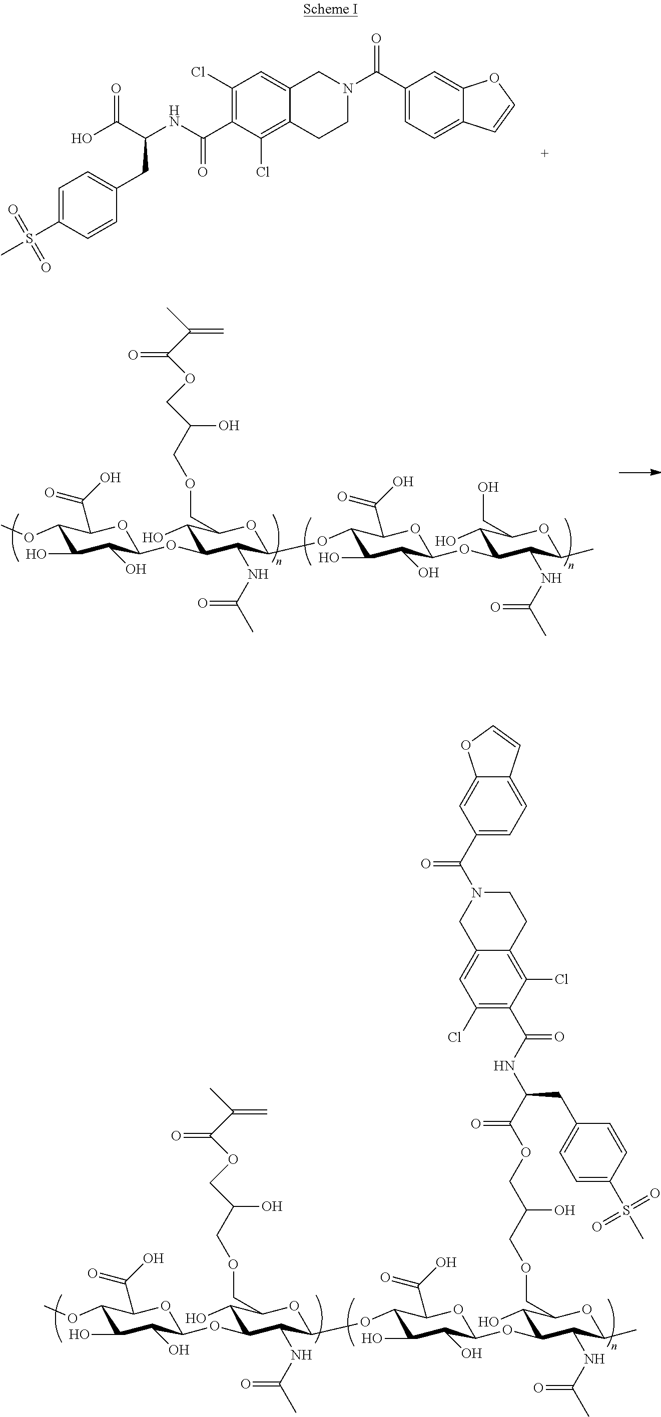

A representative example of a reaction scheme to form a lifitegrast or a salt thereof-containing hyaluronic acid polymer with a releasable linking group that is a photocleavable can be seen below in steps 1 and 2 of Scheme II.

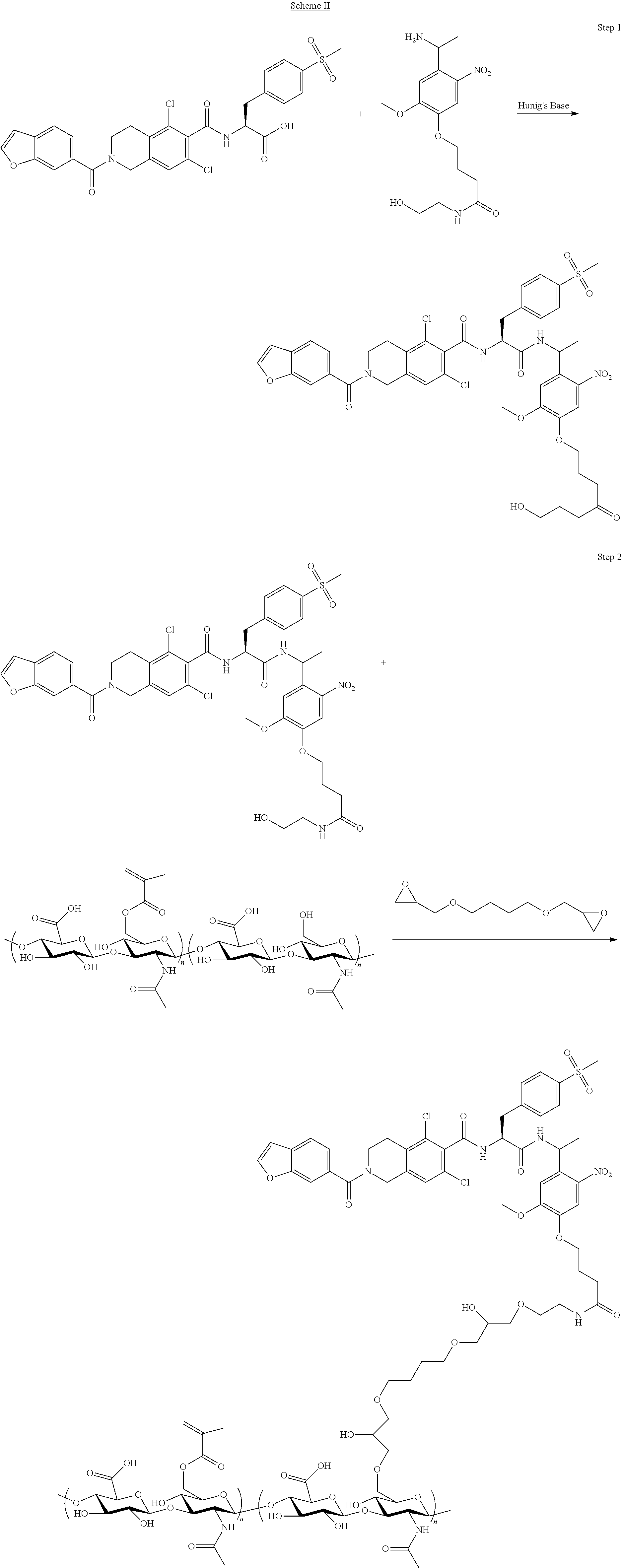

In some embodiments, the ethylenically unsaturated reactive-containing residue grafted onto a reactive functional group in the polymer backbone of the GAG is derived from a monomer comprising an ethylenically unsaturated reactive group and at least one reactive end group. In one embodiment, the ethylenically unsaturated reactive-containing residue is a methacrylate-containing residue. The at least one reactive end group includes a reactive functional group capable of grafting on to a complementary reactive functional group in the polymer backbone of the GAG. Suitable reactive functional groups of the monomer comprising an ethylenically unsaturated reactive group and at least one reactive end group include, for example, a halogen, an anhydride, an amino group, an aldehyde group, a carboxylic acid group, an alcohol group, a thiol group, a hydrazide group, a glycidyl group, etc. In one non-limiting illustrative embodiment, an ethylenically unsaturated reactive-containing residue can be derived from, for example, methacrylic anhydride, methacryloyl chloride, 2-isocyanoethylmethacrylate, 3-(trimethoxysilyl)propyl methacrylate, 3-(chlorodimethylsilyl)propyl methacrylate, glycidyl methacrylate, methacryloyl hydrazide, aminoethyl methacrylate, vinyl chloroformate, allyl chloride, acryloyl chloride, and acrylic anhydride. However, other monomers for forming the ethylenically unsaturated reactive-containing residue on the polymer backbone of the GAG are contemplated and the foregoing list is merely exemplary.

The lifitegrast or a salt thereof-containing GAG polymers disclosed herein can be obtained by grafting a reactive end group of lifitegrast or salt thereof onto a complementary reactive functionality in the polymer backbone of the GAG and grafting the at least one reactive end group of the one or more monomers comprising an ethylenically unsaturated reactive group onto another complementary reactive functionality in the polymer backbone of the GAG in any order. For example, in one illustrative embodiment, a carboxylic acid group of the lifitegrast or a salt thereof can be grafted onto a hydroxyl group in the polymer backbone of the GAG. As a further example, in one illustrative embodiment, an anhydride group of the one or more monomers comprising an ethylenically unsaturated reactive group can be grafted onto a carboxylic acid group in the polymer backbone of the GAG. As a further example, in one illustrative embodiment, an anhydride group of the one or more monomers comprising an ethylenically unsaturated reactive group can be grafted onto a hydroxyl group in the polymer backbone of the GAG and the lifitegrast or a salt thereof comprising a releasable linking group having a reactive end group can be attached onto another hydroxyl group in the polymer backbone of the GAG.

In non-limiting illustrative embodiments, the graft polymerization reaction can obtain a degree of grafting, i.e., the number of sidechains in the polymer backbone containing the ethylenically unsaturated reactive-containing residue, ranging from about 0.5 to about 50%. In another illustrative embodiment, the degree of grafting can range from about 2 to about 30%. In another illustrative embodiment, the degree of grafting can range from about 5 to about 20%. In yet another illustrative embodiment, the degree of grafting can range from about 5 to about 15%. In yet another illustrative embodiment, the degree of grafting can range from about 5 to about 10%.

In general, the GAG, lifitegrast or salt thereof, and monomer comprising an ethylenically unsaturated reactive group and at least one reactive end group can be added sequentially or simultaneously to a reaction mixture. The reaction can be carried out at a suitable temperature and for a time period for the completion of the reaction to maximize the yield of the product ethylenically unsaturated reactive-containing residue grafted onto the polymer backbone of the GAG. For example, a suitable temperature and time period includes a temperature ranging from about 10° C. to about 40° C. and a time period ranging from about 4 hours to about 48 hours. In an illustrative embodiment, a suitable temperature and time period includes a temperature ranging from about 15° C. to about 25° C. and a time period ranging from about 8 hours to about 24 hours.

In an illustrative embodiment, a GAG can be added to the reaction mixture in an amount ranging from about 5 wt. % to about 60 wt. %, based on the total weight of the reaction mixture. In one illustrative embodiment, a GAG can be added to the reaction mixture in an amount ranging from about 10 wt. % to about 50 wt. %, based on the total weight of the reaction mixture.

In an illustrative embodiment, the lifitegrast or a salt thereof can be added to the reaction mixture in an amount ranging from about 1 wt. % to about 50 wt. %, based on the total weight of the reaction mixture. In one illustrative embodiment, the lifitegrast or a salt thereof can be added to the reaction mixture in an amount ranging from about 5 wt. % to about 20 wt. %, based on the total weight of the reaction mixture.

In an illustrative embodiment, a monomer comprising an ethylenically unsaturated reactive group and at least one reactive end group can be added to the reaction mixture in an amount ranging from about 0.1 wt. % to about 5 wt. %, based on the total weight of the reaction mixture. In one illustrative embodiment, a monomer comprising an ethylenically unsaturated reactive group and at least one reactive end group can be added to the reaction mixture in an amount ranging from about 0.5 wt. % to about 2 wt. %, based on the total weight of the reaction mixture.

The resulting lifitegrast or salt thereof-containing GAG polymer can be a random copolymer or a block copolymer. In one illustrative embodiment, a lifitegrast or a salt thereof-containing GAG polymer disclosed herein can have a weight average molecular weight ranging from about 10,000 to about 6,000,000 Da in which the lower limit is from about 20,000, about 30,000, about 40,000, about 50,000, about 60,000, about 70,000, about 80,000, about 90,000, or about 100,000 Da, and the upper limit is about 100,000, about 150,000, about 200,000, about 300,000, about 400,000, about 500,000, about 600,000, about 700,000, about 800,000, about 900,000, about 1,000,000, about 2,000,000, about 3,000,000, about 4,000,000, about 5,000,000 or up to about 6,000,000 Da, wherein any of the lower limits can be combined with any of the upper limits.

In accordance with one or more additional non-limiting illustrative embodiments, as may be combined with one or more of the preceding paragraphs, the lifitegrast or a salt thereof-containing hydrophilic polymer comprising a releasable linking group and an ethylenically unsaturated reactive group can be present in the monomeric mixture in an amount ranging from about 0.1 wt. % to about 30 wt. %, based on the total weight of the monomeric mixture. In some embodiments, the lifitegrast or a salt thereof-containing hydrophilic polymer comprising a releasable linking group and an ethylenically unsaturated reactive group can be present in the monomeric mixture in an amount ranging from about 1 wt. % to about 10 wt. %, based on the total weight of the monomeric mixture.

In non-limiting illustrative embodiments, as may be combined with one or more of the preceding paragraphs, the monomeric mixture further includes one or more ophthalmic device-forming comonomers. In some embodiments, the one or more ophthalmic device-forming comonomers can be present in the monomeric mixture in an amount ranging from about 0.1 wt. % to about 98 wt. %, based on the total weight of the monomeric mixture. In some embodiments, the one or more ophthalmic device-forming comonomers can be present in the monomeric mixture in an amount ranging from about 0.1 wt. % to about 90 wt. %, based on the total weight of the monomeric mixture.

In an embodiment, the one or more ophthalmic device-forming comonomers can be any material not hydrophilic per se. These materials are known in the art and include, by way of example, polysiloxanes, perfluoropolyethers, fluorinated poly(meth)acrylates or equivalent fluorinated polymers derived, e.g., from other polymerizable carboxylic acids, polyalkyl(meth)acrylates or equivalent alkylester polymers derived from other polymerizable carboxylic acids, or fluorinated polyolefins, such as fluorinated ethylene propylene polymers, or tetrafluoroethylene, preferably in combination with a dioxol, e.g., perfluoro-2,2-dimethyl-1,3-dioxol.

In some embodiments, the one or more ophthalmic device-forming comonomers include one or more hydrophilic comonomers. Suitable one or more hydrophilic comonomers include, for example, unsaturated carboxylic acids, acrylamides, vinyl lactams, hydroxyl-containing-(meth)acrylates, hydrophilic vinyl carbonates, hydrophilic vinyl carbamates, hydrophilic oxazolones, and poly(alkene glycols) functionalized with polymerizable groups and the like and mixtures thereof.

Representative examples of unsaturated carboxylic acids include, but are not limited to, methacrylic acid, acrylic acid and the like and mixtures thereof. Representative examples of acrylamides include, but are not limited to, alkylamides such as N,N-dimethylacrylamide, N,N-dimethylmethacrylamide and the like and mixtures thereof. Representative examples of cyclic lactams include, but are not limited to, N-vinyl-2-pyrrolidone, N-vinyl caprolactam, N-vinyl-2-piperidone and the like and mixtures thereof. Representative examples of hydroxyl-containing (meth)acrylates include, but are not limited to, 2-hydroxyethyl methacrylate (HEMA), glycerol methacrylate and the like and mixtures thereof. Additional device-forming hydrophilic comonomers include, for example, the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable device-forming hydrophilic comonomers will be apparent to one skilled in the art. Mixtures of the foregoing device-forming hydrophilic comonomers can also be used in the silicone contact lens-forming mixtures herein.

The monomeric mixture may further include a second ophthalmic device-forming comonomer including, for example, a copolymerizable group and a reactive functional group. The copolymerizable group is preferably an ethylenically unsaturated group, such that this ophthalmic device-forming comonomer copolymerizes with the hydrophilic monomer and any other ophthalmic device-forming comonomers in the initial monomeric mixture. Additionally, the second monomer can include a reactive functional group that reacts with a complementary reactive group of the lifitegrast or a salt thereof-containing hydrophilic polymer.

In an embodiment, reactive groups of the second ophthalmic device-forming comonomers include epoxide groups. Accordingly, second ophthalmic device-forming comonomers are those that include both an ethylenically unsaturated group (that permits the monomer to copolymerize with the hydrophilic monomer) and the epoxide group (that does not react with the hydrophilic monomers but remains to react with the lifitegrast or a salt thereof-containing hydrophilic polymer, e.g., the reaction product of one or more polymerizable polyhydric alcohols and one or more polymerizable fluorine-containing monomers). Suitable second ophthalmic device-forming comonomers include, for example, glycidyl methacrylate, glycidyl acrylate, glycidyl vinylcarbonate, glycidyl vinylcarbamate, and 4-vinyl-1-cyclohexene-1,2-epoxide.

It is particularly useful to employ biocompatible materials herein including both soft and rigid materials commonly used for contact lenses. In general, non-hydrogel materials are hydrophobic polymeric materials that do not contain water in their equilibrium state. Typical non-hydrogel materials comprise silicone acrylics, such as those formed from a bulky silicone monomer (e.g., tris(trimethylsiloxy)silylpropyl methacrylate, commonly known as “TRIS” monomer), methacrylate end-capped poly(dimethylsiloxane)prepolymer, or silicones having fluoroalkyl side groups (polysiloxanes are also commonly known as silicone polymers).

In another embodiment, the ophthalmic devices such as contact lenses formed from the monomeric mixtures disclosed herein can be a hydrogel. Hydrogels in general are a well-known class of materials that comprise hydrated, crosslinked polymeric systems containing water in an equilibrium state. Accordingly, hydrogels are copolymers prepared from hydrophilic monomers as discussed above. In the case of silicone hydrogels, the hydrogel copolymers are generally prepared by polymerizing a mixture containing at least one contact lens-forming silicone-containing monomer and at least one contact lens-forming hydrophilic monomer. Either the silicone-containing monomer or the hydrophilic monomer can function as a crosslinking agent (a crosslinker being defined as a monomer having multiple polymerizable functionalities) or a separate crosslinker may be employed. Silicone hydrogels typically have a water content between about 10 wt. % to about 80 wt. %.

As mentioned, one class of ophthalmic device materials are silicone hydrogels. In this case, the initial monomeric mixture further includes a silicone-containing monomer. Applicable silicone-containing monomers for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779; and 5,358,995. Specific examples of suitable materials for use herein include those disclosed in U.S. Pat. Nos. 5,310,779; 5,387,662; 5,449,729; 5,512,205; 5,610,252; 5,616,757; 5,708,094; 5,710,302; 5,714,557 and 5,908,906, the contents of which are incorporated by reference herein.

In some embodiments, a silicone-containing monomer includes a bulky siloxane monomer having an ethylenically unsaturated reactive group. The term “bulky” refers to groups on the siloxane monomer that are sterically and/or electronically encumbering, i.e., sterically hindering. In a non-limiting illustrative embodiment, suitable bulky siloxane monomers include, for example, a bulky polysiloxanylalkyl (meth)acrylic monomer, a bulky polysiloxanylalkyl carbamate monomer and mixtures thereof. In one embodiment, a representative example of a bulky siloxane monomer is represented by a structure of Formula Ia:

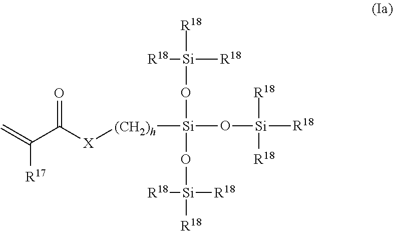

wherein X denotes —O— or —NR¹⁹—, where each R¹⁹ is hydrogen or a C₁-C₄ alkyl group; R¹⁷ independently denotes hydrogen or methyl; each R¹⁸ independently denotes a lower alkyl radical such as a C₁-C₆ group, a phenyl radical or a group represented by the following structure:

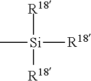

wherein each R^{18′} independently denotes a lower alkyl radical such as a C₁-C₆ group or a phenyl radical; and h is 1 to 10; or is represented by a structure of Formula Ib:

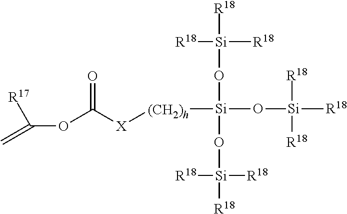

wherein X denotes —NR¹⁹— wherein R¹⁹ denotes hydrogen or a C₁-C₄ alkyl; R¹⁷ denotes hydrogen or methyl; each R⁸ independently denotes a lower alkyl radical such as a C₁-C₆ group, a phenyl radical or a group represented by the following structure:

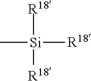

wherein each R^{18′} independently denotes a lower alkyl radical such as a C₁-C₆ group or a phenyl radical; and h is 1 to 10.

Representative examples of bulky siloxane monomers include 3-methacryloyloxypropyltris(trimethylsiloxy)silane or tris(trimethylsiloxy)silylpropyl methacrylate, sometimes referred to as TRIS and tris(trimethylsiloxy)silylpropyl vinyl carbamate, sometimes referred to as TRIS-VC, pentamethyldisiloxanyl methylmethacrylate, phenyltetramethyl-disiloxanylethyl acetate, and methyldi(trimethylsiloxy)methacryloxymethyl silane, (3-methacryloxy-2-hydroxy propoxy)propyl bis(trimethyl siloxy)methyl silane, sometimes referred to as Sigma and the like and mixtures thereof. In one embodiment, the bulky siloxane monomer is a tris(trialkylsiloxy)silylalkyl methacrylate-containing monomer such as a tris(trimethylsiloxy)silylpropyl methacrylate-containing monomer.

Such bulky monomers may be copolymerized with a silicone macromonomer, which is a poly(organosiloxane) capped with an unsaturated group at two or more ends of the molecule. U.S. Pat. No. 4,153,641 discloses, for example, various unsaturated groups such as acryloxy or methacryloxy groups.

Another class of representative silicone-containing monomers includes, for example, silicone-containing vinyl carbonate or vinyl carbamate monomers such as, for example, 1,3-bis[4-vinyloxycarbonyloxy)but-1-yl]tetramethyl-disiloxane; 3-(trimethylsilyl)propyl vinyl carbonate; 3-(vinyloxycarbonylthio)propyl-[tris(trimethylsiloxy)silane]; 3-[tris(trimethylsiloxy)silyl]propyl vinyl carbamate; 3-[tris(trimethylsiloxy)silyl]propyl allyl carbamate; 3-[tris(trimethylsiloxy)silyl]propyl vinyl carbonate; t-butyldimethylsiloxyethyl vinyl carbonate; trimethylsilylethyl vinyl carbonate; trimethylsilylmethyl vinyl carbonate and the like and mixtures thereof.

Another class of silicone-containing monomers includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formula II and III:

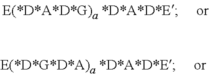

wherein:
D independently denotes an alkyl diradical, an alkyl cycloalkyl diradical, a cycloalkyl diradical, an aryl diradical or an alkylaryl diradical having 6 to about 30 carbon atoms;
G independently denotes an alkyl diradical, a cycloalkyl diradical, an alkyl cycloalkyl diradical, an aryl diradical or an alkylaryl diradical having 1 to about 40 carbon atoms and which may contain ether, thio or amine linkages in the main chain;
* denotes a urethane or ureido linkage;
a is at least 1;
A independently denotes a divalent polymeric radical of Formula (IV):

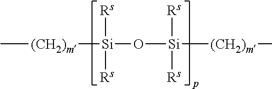

wherein each R^{s} independently denotes an alkyl or fluoro-substituted alkyl group having 1 to about 10 carbon atoms which may contain ether linkages between the carbon atoms; m′ is at least 1; and p is a number that provides a moiety weight of about 400 to about 10,000;
each of E and E′ independently denotes a polymerizable unsaturated organic radical represented by Formula V:

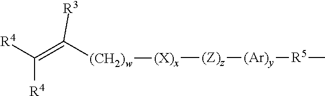

wherein: R³ is hydrogen or methyl;
R⁴ is hydrogen, an alkyl radical having 1 to 6 carbon atoms, or a —CO—Y—R⁶ radical wherein Y is —O—, —S— or —NH;
R⁵ is a divalent alkylene radical having 1 to about 10 carbon atoms;
R⁶ is an alkyl radical having 1 to about 12 carbon atoms;
X denotes —CO— or —OCO—;
Z denotes —O— or —NH—;
Ar denotes an aromatic radical having about 6 to about 30 carbon atoms;
w is 0 to 6; x is 0 or 1; y is 0 or 1; and z is 0 or 1.

In an embodiment, a silicone-containing urethane monomer is represented by Formula VI:

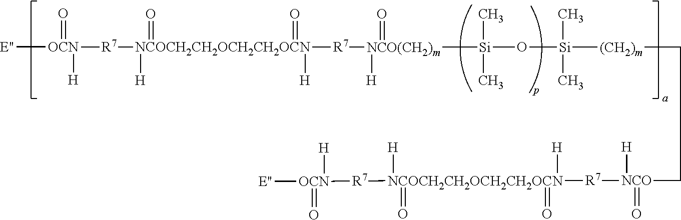

wherein m is at least 1 and is preferably 3 or 4, a is at least 1 and preferably is 1, p is a number which provides a moiety weight of about 400 to about 10,000 and is preferably at least about 30, R⁷ is a diradical of a diisocyanate after removal of the isocyanate group, such as the diradical of isophorone diisocyanate, and each E″ is a group represented by:

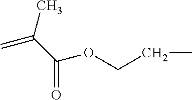

In another embodiment, a silicone hydrogel material comprises (in bulk, that is, in the monomer mixture that is copolymerized) about 5 wt. % to about 50 wt. %, or from about 10 wt. % to about 25 wt. % of one or more silicone macromonomers, about 5 wt. % to about 75 wt. %, or about 30 wt. % to about 60 wt. % of one or more polysiloxanylalkyl (meth)acrylic monomers, and about 10 wt. % to about 50 wt. %, or about 20 wt. % to about 40 wt. % of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 discloses additional unsaturated groups, including acryloxy or methacryloxy. Fumarate-containing materials such as those disclosed in U.S. Pat. Nos. 5,310,779; 5,449,729 and 5,512,205 are also useful substrates in accordance with the invention. The silane macromonomer may be a silicon-containing vinyl carbonate or vinyl carbamate or a polyurethane-polysiloxane having one or more hard-soft-hard blocks and end-capped with a hydrophilic monomer.

In another embodiment, a class of silicon-containing monomers includes monomers of Formula VII:

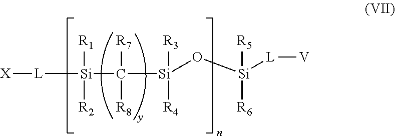

wherein X is the residue of a ring opening agent; L is the same or different and is a linker group or a bond; V is an ethylenically unsaturated polymerizable group; R₁, R₂, R₃, R₄, R₅, R₆ are independently H, alkyl, halo alkyl, cyclo alkyl, heterocyclo alkyl, alkenyl, halo alkenyl, or aromatic; R₇ and R₈ are independently H or alkyl wherein at least one of R₇ or R₈ is hydrogen; y is 2 to 7 and n is 1 to 100.

Ring opening agents are well known in the literature. Non-limiting examples of anionic ring opening agents include alkyl lithiums, alkoxides, trialkylsiloxylithium wherein the alkyl group may or may not contain halo atoms.

Linker groups can be any divalent radical or moiety and include substituted or unsubstituted alkyl, alkyl ether, alkenyls, alkenyl ethers, halo alkyls, substituted or unsubstituted siloxanes, and monomers capable of propagating ring opening.

Ethylenically unsaturated polymerizable groups are well known to those skilled in the art. Non-limiting examples of ethylenically unsaturated polymerizable groups would include acrylates, methacrylates, vinyl carbonates, O-vinyl carbamates, N-vinyl carbamates, acrylamides and methacrylamides.

In another embodiment, a class of silicone-containing monomers includes monomers of Formula VIII:

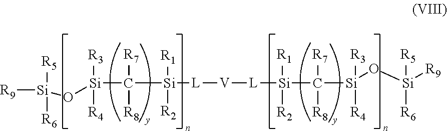

wherein L is the same or different and is a linker group or a bond; V is the same or different and is an ethylenically unsaturated polymerizable group; R₁, R₂, R₃, R₄, R₅, R₆ and R₉ are independently H, alkyl, halo alkyl, cyclo alkyl, heterocyclo alkyl, alkenyl, halo alkenyl, or aromatic; R₇ and R₈ are independently H or alkyl wherein at least one of R₇ or R₈ is hydrogen; y is 2-7 and n is 1-100.

In another embodiment, a class of silicone-containing monomers includes monomers of Formulas IX and X:

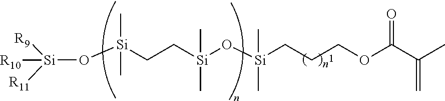

wherein R₉, R₁₀ and R₁₁ are independently H, alkyl, haloalkyl or other substituted alkyl groups; n is as defined above and n¹ is 0-10; and,

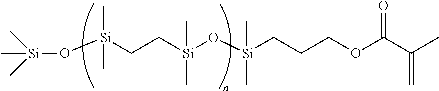

wherein n is 1 to 100, or n is 2 to 80, or n is 3 to 20, or n is 5 to 15.

In another embodiment, a class of silicon-containing monomers includes monomers of Formulas XI-XV:

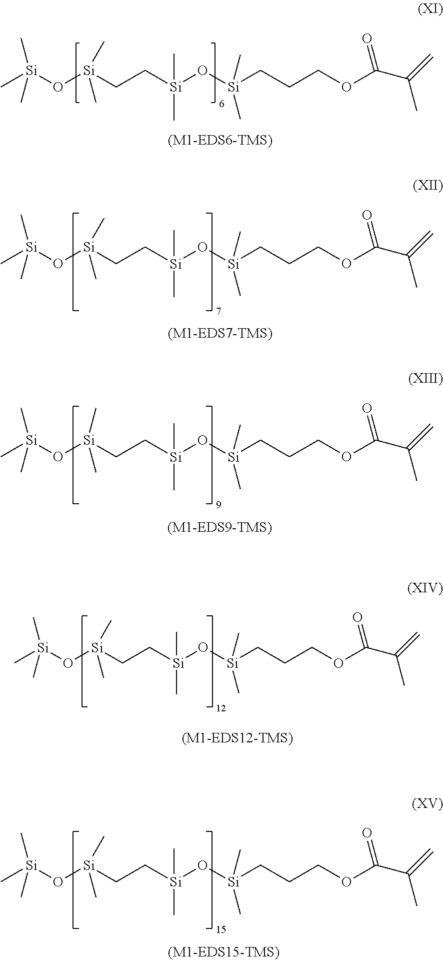

In another embodiment, a class of silicone-containing monomers includes monomers of Formulas XVI-XVIII:

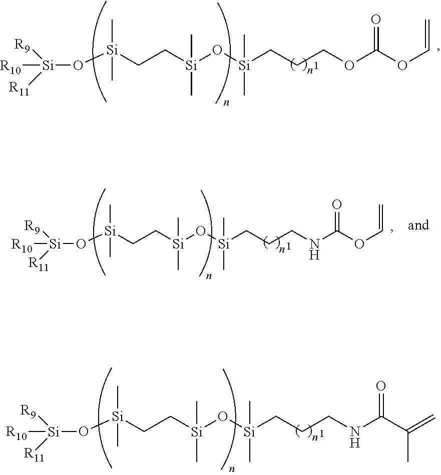

wherein R₉, R₁₀ and R₁₁ are independently H, alkyl, haloalkyl or other substituted alkyl groups and n and n¹ are as defined above.

In another embodiment, a class of silicone-containing monomers includes monomers of Formulas XIX-XXI:

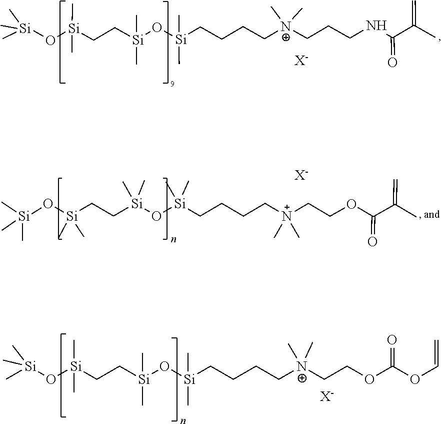

wherein n is as defined above and X⁻ is a counterion to provide an overall neutral charge.

Counterions capable of providing an overall neutral charge are well known to those of ordinary skill in the art and would include, for example, halide ions.

In another embodiment, a class of silicone-containing monomers includes monomers of Formula XXII:

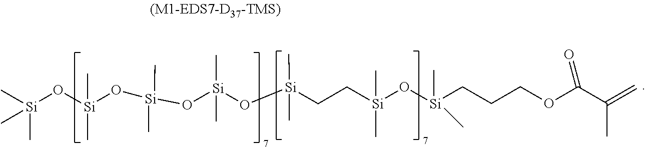

Another class of representative silicone-containing monomers includes fluorinated monomers. Such monomers have been used in the formation of fluorosilicone hydrogels to reduce the accumulation of deposits on contact lenses made therefrom, as disclosed in, for example, U.S. Pat. Nos. 4,954,587; 5,010,141 and 5,079,319. Also, the use of silicone-containing monomers having certain fluorinated side groups, i.e., —(CF₂)—H, have been found to improve compatibility between the hydrophilic and silicone-containing monomeric units. See, e.g., U.S. Pat. Nos. 5,321,108 and 5,387,662.

The above silicone materials are merely exemplary, and other materials for use as ophthalmic devices that have been disclosed in various publications and are being continuously developed for use in contact lenses and other ophthalmic devices can also be used. For example, a contact lens can be formed from at least a cationic monomer such as cationic silicone-containing monomer or cationic fluorinated silicone-containing monomers.

In non-limiting illustrative embodiments, as may be combined with one or more of the preceding paragraphs, the monomeric mixtures can further contain a reactive (polymerizable) ultraviolet (UV) light absorber and/or a reactive blue-light absorber. Suitable reactive UV light absorbers can be any known reactive UV light absorber. In non-limiting illustrative embodiments, suitable reactive UV light absorbers include, for example, 2-(2′-hydroxy-3′-methallyl-5′-methylphenyl)benzotriazole, commercially available as o-Methallyl Tinuvin P (“oMTP”) from Polysciences, Inc., Warrington, Pa., 3-(2H-benzo[d][1,2,3]triazol-2-yl)-4-hydroxyphenylethyl methacrylate, and 2-(3-(tert-butyl)-4-hydroxy-5-(5-methoxy-2H-benzo[d][1,2,3]triazol-2-yl)phenoxy)ethyl methacrylate.

In one illustrative embodiment, suitable UV light absorbers include, for example, one or more compounds of the following formulas:

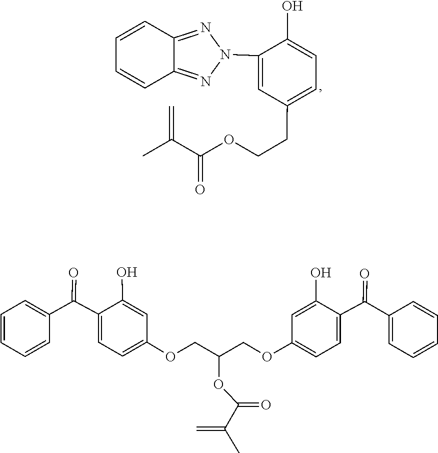 [SMILES: C=C(C)C(=O)OCCc1ccc(O)c(-n2nc3ccccc3n2)c1 ; C=C(C)C(=O)OC(COc1ccc(C(=O)c2ccccc2)c(O)c1)COc1ccc(C(=O)c2ccccc2)c(O)c1]

(2-Propenoic acid, 2-methyl,2-(4-benzoyl-3-hydroxyphenoxy)-1-[(4-benzoyl3-hydroxyphenoxy)methyl ester),

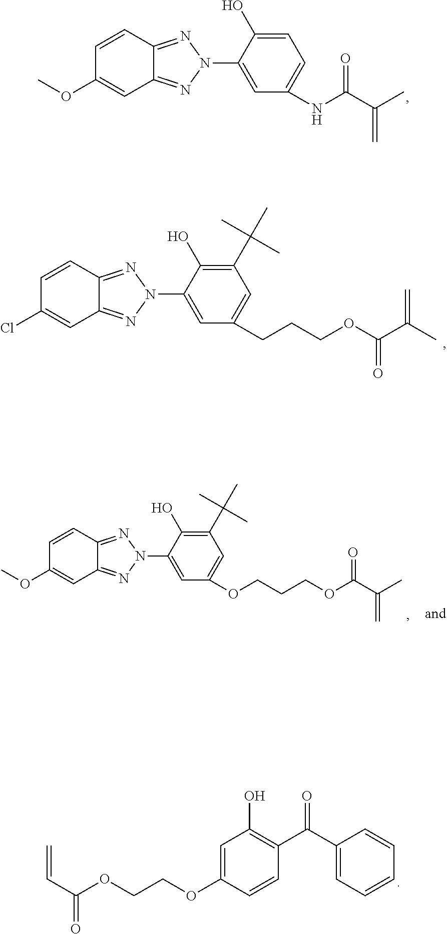

These compounds are merely illustrative and not intended to be limiting. Any known UV blocker or later developed UV blocker is contemplated for use herein.

In illustrative embodiments, the UV light absorbers can be present in the monomeric mixture in an amount ranging from about 0.1 wt. % to about 5 wt. %, based on the total weight of the monomeric mixture. In another illustrative embodiment, the UV light absorbers can be present in the monomeric mixture in an amount ranging from about 1.5 wt. % to about 2.5 wt. %, based on the total weight of the monomeric mixture. In yet another non-limiting illustrative embodiment, the UV light absorbers can be present in the monomeric mixture in an amount ranging from about 1.5 wt. % to about 2 wt. %, based on the total weight of the monomeric mixture.

Many reactive blue-light absorbing compounds are known. Preferred reactive blue-light absorbing compounds are those described in U.S. Pat. Nos. 5,470,932; 8,207,244; and 8,329,775, the contents of which are hereby incorporated by reference. In one embodiment, a blue-light absorbing dye is N-2-[3-(2′-methylphenylazo)-4-hydroxyphenyl]ethyl methacrylamide. In illustrative embodiments, the blue-light absorbers can be present in the silicone hydrogel contact lens-forming mixture in an amount ranging from about 0.005 wt. % to about 1 wt. %, based on the total weight of the monomeric mixture. In another illustrative embodiment, the blue-light absorbers can be present in the monomeric mixture in an amount ranging from about 0.01 wt. % to about 1 wt. %, based on the total weight of the monomeric mixture.

The ophthalmic devices of the illustrative embodiments can be prepared by polymerizing the foregoing monomeric mixtures to form a product that can be subsequently formed into the appropriate shape by, for example, lathing, injection molding, compression molding, cutting and the like. For example, in producing contact lenses, the initial monomeric mixture may be polymerized in tubes to provide rod-shaped articles, which are then cut into buttons. The buttons may then be lathed into contact lenses.

Alternately, the ophthalmic devices may be cast directly in molds, e.g., polypropylene molds, from the mixtures, e.g., by spincasting and static casting methods. Spincasting methods are disclosed in U.S. Pat. Nos. 3,408,429 and 3,660,545, and static casting methods are disclosed in U.S. Pat. Nos. 4,113,224, 4,197,266, and 5,271,875. Spincasting methods involve charging the mixtures to be polymerized to a mold, and spinning the mold in a controlled manner while exposing the mixture to a radiation source such as UV light. Static casting methods involve charging the monomeric mixture between two mold sections, one mold section shaped to form the anterior lens surface and the other mold section shaped to form the posterior lens surface, and curing the mixture while retained in the mold assembly to form a lens, for example, by free radical polymerization of the mixture.

Examples of free radical reaction techniques to cure the ophthalmic device material include thermal radiation, infrared radiation, electron beam radiation, gamma radiation, ultraviolet (UV) radiation, and the like; or combinations of such techniques may be used. U.S. Pat. No. 5,271,875 describes a static cast molding method that permits molding of a finished lens in a mold cavity defined by a posterior mold and an anterior mold. As an additional method, U.S. Pat. No. 4,555,732 discloses a process where an excess of a mixture is cured by spincasting in a mold to form a shaped article having an anterior lens surface and a relatively large thickness, and the posterior surface of the cured spincast article is subsequently lathed to provide a contact lens having the desired thickness and posterior lens surface.

Polymerization may be facilitated by exposing the mixture to heat and/or radiation, such as ultraviolet light, visible light, or high energy radiation. A polymerization initiator may be included in the mixture to facilitate the polymerization step. Representative examples of free radical thermal polymerization initiators include organic peroxides such as acetyl peroxide, lauroyl peroxide, decanoyl peroxide, stearoyl peroxide, benzoyl peroxide, tertiarylbutyl peroxypivalate, peroxydicarbonate, and the like. Representative UV initiators are those known in the art and include benzoin methyl ether, benzoin ethyl ether, Darocure® 1173, 1164, 2273, 1116, 2959, 3331 (EM Industries) and Irgacure® 651, 184 and 2959 (Ciba-Geigy), 2,2′Azobis(2-methylpropionitrile) (VAZO 64) and the like. Generally, the initiator will be employed in the mixture at a concentration of about 0.01 to about 5 percent by weight of the total mixture.

Polymerization is generally performed in a reaction medium, such as, for example, a solution or dispersion using a solvent, e.g., water or an alkanol containing from 1 to 4 carbon atoms such as methanol, ethanol or propan-2-ol. Alternatively, a mixture of any of the above solvents may be used.

Generally, polymerization can be carried out for about 15 minutes to about 72 hours, and under an inert atmosphere of, for example, nitrogen or argon. If desired, the resulting polymerization product can be dried under vacuum, e.g., for about 5 hours to about 72 hours or left in an aqueous solution prior to use.

Polymerization of the monomeric mixtures will yield a polymerization product, that when hydrated, preferably forms a hydrogel. When producing a hydrogel lens, the monomeric mixture may further include at least a diluent that is ultimately replaced with water when the polymerization product is hydrated to form a hydrogel. The amount of diluent used should be less than about 50 wt. %, and in most cases, the diluent content will be less than about 30 wt. %. However, in a particular polymer system, the actual limit will be dictated by the solubility of the various monomers in the diluent. In order to produce an optically clear copolymer, it is important that a phase separation leading to visual opacity does not occur between the comonomers and the diluent, or the diluent and the final copolymer.

Furthermore, the maximum amount of diluent which may be used will depend on the amount of swelling the diluent causes the final polymers. Excessive swelling will or may cause the copolymer to collapse when the diluent is replaced with water upon hydration. Suitable diluents include, but are not limited to, ethylene glycol, glycerine, liquid poly(ethylene glycol), alcohols, alcohol/water mixtures, ethylene oxide/propylene oxide block copolymers, low molecular weight linear poly(2-hydroxyethyl methacrylate), glycol esters of lactic acid, formamides, ketones, dialkylsulfoxides, butyl carbitol, and the like and mixtures thereof.

If necessary, it may be desirable to remove residual diluent from the ophthalmic device before edge-finishing operations which can be accomplished by evaporation at or near ambient pressure or under vacuum. An elevated temperature can be employed to shorten the time necessary to evaporate the diluent. The time, temperature and pressure conditions for the solvent removal step will vary depending on such factors as the volatility of the diluent and the specific monomeric components, as can be readily determined by one skilled in the art. If desired, the mixture used to produce the hydrogel lens may further include crosslinking and wetting agents known in the prior art for making hydrogel materials.

The ophthalmic devices obtained herein may be subjected to optional machining operations. For example, the optional machining steps may include buffing or polishing a lens edge and/or surface. Generally, such machining processes may be performed before or after the product is released from a mold part, e.g., the lens is dry released from the mold by employing vacuum tweezers to lift the lens from the mold, after which the lens is transferred by means of mechanical tweezers to a second set of vacuum tweezers and placed against a rotating surface to smooth the surface or edges. The lens may then be turned over in order to machine the other side of the lens.

The ophthalmic device may then be transferred to individual lens packages containing a buffered saline solution. The saline solution may be added to the package either before or after transfer of the lens. Appropriate packaging designs and materials are known in the art. A plastic package is releasably sealed with a film. Suitable sealing films are known in the art and include foils, polymer films and mixtures thereof. The sealed packages containing the lenses are then sterilized to ensure a sterile product. Suitable sterilization means and conditions are known in the art and include, for example, autoclaving.

As one skilled in the art will readily appreciate other steps may be included in the molding and packaging process described above. Such other steps can include, for example, coating the formed lens, surface treating the lens during formation (e.g., via mold transfer), inspecting the lens, discarding defective lenses, cleaning the mold halves, reusing the mold halves, and the like and combinations thereof.

The following examples are provided to enable one skilled in the art to practice the invention and are merely illustrative of the invention. The examples should not be read as limiting the scope of the invention as defined in the claims.

In the examples, the following abbreviations are used.
DMA: N,N-dimethylacrylamide.
HEMA: 2-hydroxyethyl methacrylate.
EGDMA: Ethylene glycol dimethacrylate.
HEMA-lifitegrast: A monomer having the following structure:

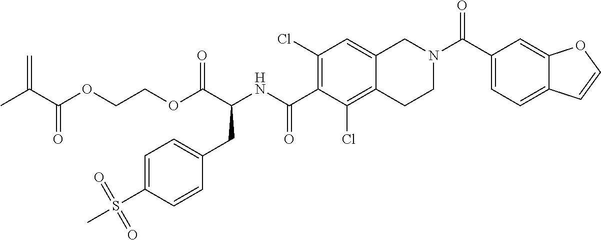 [SMILES: C=C(C)C(=O)OCCOC(=O)[C@H](Cc1ccc(S(C)(=O)=O)cc1)NC(=O)c1c(Cl)cc2c(c1Cl)CCN(C(=O)c1ccc3ccoc3c1)C2]

### Example 1

### Preparation of a Contact Lens.

A contact lens is prepared using the reaction components listed in Table 1 below, as amounts per weight percent.

TABLE 1

| Component | (Wt. %) |
|---|---|
| HEMA | 48.00 |
| DMA | 49.70 |
| EGDMA | 0.20 |
| Initiator | 0.10 |
| HA-Lifitegrast | 2.00 |

According to an aspect of the present disclosure, an ophthalmic device comprises a polymerization product of a monomeric mixture comprising (a) a lifitegrast or a salt thereof-containing hydrophilic polymer comprising a releasable linking group and an ethylenically unsaturated reactive group; and (b) an ophthalmic device-forming comonomer.

In non-limiting illustrative embodiments, as may be combined with one or more of the preceding paragraphs, the lifitegrast or a salt thereof-containing hydrophilic polymer comprises a lifitegrast or a salt thereof-containing hyaluronic acid polymer.

In non-limiting illustrative embodiments, as may be combined with one or more of the preceding paragraphs, the lifitegrast or a salt thereof-containing hydrophilic polymer comprises lifitegrast or a salt thereof attached to the hydrophilic polymer via the releasable linking group.

In non-limiting illustrative embodiments, as may be combined with one or more of the preceding paragraphs, the releasable linking group attached to the lifitegrast or a salt thereof and the hydrophilic polymer is one of a pH-labile bond, an acid-labile bond, a base-labile bond, an oxidatively labile bond, a metabolically labile bond, a biochemically labile bond, an enzyme-labile bond, a chemical-labile bond or a photo-labile bond.

In non-limiting illustrative embodiments, as may be combined with one or more of the preceding paragraphs, the lifitegrast or a salt thereof-containing hydrophilic polymer comprises a lifitegrast or a salt thereof-containing glycosaminoglycan polymer.

In non-limiting illustrative embodiments, as may be combined with one or more of the preceding paragraphs, the lifitegrast or a salt thereof-containing glycosaminoglycan polymer comprises a glycosaminoglycan having a polymer backbone and a first side chain comprising a lifitegrast or a salt thereof residue comprising the releasable linking group grafted onto the polymer backbone, and a second side chain comprising an ethylenically unsaturated reactive-containing residue grafted onto the polymer backbone.

In non-limiting illustrative embodiments, as may be combined with one or more of the preceding paragraphs, the ethylenically unsaturated reactive group comprises a (meth)acrylate group.

In non-limiting illustrative embodiments, as may be combined with one or more of the preceding paragraphs, the ophthalmic device-forming comonomer comprises one or more hydrophilic monomers.

In non-limiting illustrative embodiments, as may be combined with one or more of the preceding paragraphs, the one or more hydrophilic monomers are selected from the group consisting of an amide, cyclic lactam, hydroxyl-containing (meth)acrylate, poly(alkene glycols) functionalized with polymerizable groups and mixtures thereof.

In non-limiting illustrative embodiments, as may be combined with one or more of the preceding paragraphs, the amide is selected from the group consisting of N,N-dimethylacrylamide, N,N-dimethylmethacrylamide and mixtures thereof.

In non-limiting illustrative embodiments, as may be combined with one or more of the preceding paragraphs, the cyclic lactam is selected from the group consisting of N-vinyl-2-pyrrolidone, N-vinyl caprolactam, N-vinyl-2-piperidone and mixtures thereof.

In non-limiting illustrative embodiments, as may be combined with one or more of the preceding paragraphs, the ophthalmic device-forming comonomer comprises one or more silicone monomers.

In non-limiting illustrative embodiments, as may be combined with one or more of the preceding paragraphs, the one or more silicone monomers comprise a non-bulky organosilicon-containing monomer.

In non-limiting illustrative embodiments, as may be combined with one or more of the preceding paragraphs, the non-bulky organosilicon-containing monomer is represented by the following structure:

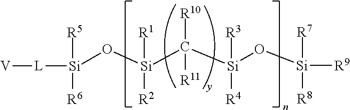

wherein V is ethylenically unsaturated polymerizable group, L is a linker group or a bond; R¹, R², R³, R⁴, R⁵, R⁶, R⁷, R⁸, and R⁹ are independently hydrogen an alkyl group, a haloalkyl group, a cycloalkyl group, a heterocycloalkyl group, an alkenyl group, a halo alkenyl group, or an aromatic group; R¹⁰ and R¹¹ are independently hydrogen or alkyl wherein at least one of R¹⁰ and R¹¹ is hydrogen; y is 2 to 7 and n is 1 to 100, or the non-bulky organosilicon-containing monomer is represented by the following structure:

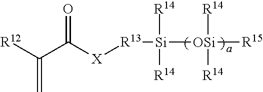

wherein R¹² is H or methyl; X is O or NR¹⁶; wherein R¹⁶ is hydrogen or C₁ to C₄ alkyl, which may be further substituted with one or more hydroxyl groups; R¹³ is a divalent alkyl group, which may further be functionalized with a group selected from the group consisting of an ether group, a hydroxyl group, a carbamate group and combinations thereof; each R¹⁴ is independently a phenyl or C₁ to C₄ alkyl which may be substituted with fluorine, hydroxyl or an ether; R¹⁵ is a C₁ to C₄ alkyl; and a is 2 to 50.

In non-limiting illustrative embodiments, as may be combined with one or more of the preceding paragraphs, the one or more silicone monomers comprise a bulky siloxane monomer.

In non-limiting illustrative embodiments, as may be combined with one or more of the preceding paragraphs, the bulky siloxane monomer is selected from the group consisting of a bulky polysiloxanylalkyl (meth)acrylic monomer, a bulky polysiloxanylalkyl carbamate monomer and mixtures thereof.

In non-limiting illustrative embodiments, as may be combined with one or more of the preceding paragraphs, the bulky siloxane monomer is represented by the following structure:

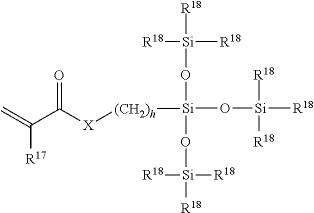

wherein X denotes —O— or —NR¹⁹— where each R¹⁹ is hydrogen or a C₁-C₄ alkyl; R¹⁷ independently denotes hydrogen or methyl; each R¹⁸ independently denotes a lower alkyl radical, a phenyl radical or a group represented by the following structure:

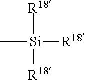

wherein each R^{18′} independently denotes a lower alkyl radical or a phenyl radical; and h is 1 to 10; or the following structure:

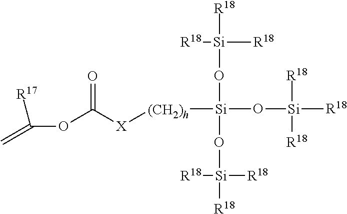

wherein X denotes —NR¹⁹⁻; wherein R¹⁹ denotes hydrogen or a C₁-C₄ alkyl; R¹⁷ denotes hydrogen or methyl; each R⁸ independently denotes a lower alkyl radical, a phenyl radical or a group represented by the following structure:

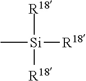

wherein each R^{18′} independently denotes a lower alkyl radical or a phenyl radical; and h is 1 to 10.

In non-limiting illustrative embodiments, as may be combined with one or more of the preceding paragraphs, the monomeric mixture further comprises an ultraviolet blocker.

In non-limiting illustrative embodiments, as may be combined with one or more of the preceding paragraphs, the monomeric mixture further comprises a blue light blocker.

In non-limiting illustrative embodiments, as may be combined with one or more of the preceding paragraphs, the lifitegrast or a salt thereof-containing hydrophilic polymer is present in the monomeric mixture in an amount ranging from about 0.1 wt. % to about 30 wt. %, based on the total weight of the monomeric mixture.

In non-limiting illustrative embodiments, as may be combined with one or more of the preceding paragraphs, the lifitegrast or a salt thereof-containing hydrophilic polymer is present in the monomeric mixture in an amount ranging from about 1 wt. % to about 10 wt. %, based on the total weight of the monomeric mixture.

In non-limiting illustrative embodiments, as may be combined with one or more of the preceding paragraphs, the ophthalmic device-forming comonomer is present in the monomeric mixture in an amount ranging from about 0.1 wt. % to about 98 wt. %, based on the total weight of the monomeric mixture.

In non-limiting illustrative embodiments, as may be combined with one or more of the preceding paragraphs, the ophthalmic device-forming comonomer is present in the monomeric mixture in an amount ranging from about 0.1 wt. % to about 90 wt. %, based on the total weight of the monomeric mixture.

In non-limiting illustrative embodiments, as may be combined with one or more of the preceding paragraphs, the ophthalmic device is a contact lens.

In non-limiting illustrative embodiments, as may be combined with one or more of the preceding paragraphs, the contact lens is a soft contact lens.

In non-limiting illustrative embodiments, as may be combined with one or more of the preceding paragraphs, the lifitegrast or a salt thereof is released in a sustained release manner.

According to another aspect of the present disclosure, a method for making an ophthalmic device, comprising:
(a) subjecting a monomeric mixture comprising (i) a lifitegrast or a salt thereof-containing hydrophilic polymer comprising a releasable linking group and an ethylenically unsaturated reactive group, and (ii) an ophthalmic device-forming comonomer to polymerization conditions to provide a polymerized ophthalmic device, and
(b) hydrating the polymerized ophthalmic device.

In non-limiting illustrative embodiments, as may be combined with one or more of the preceding paragraphs, the lifitegrast or a salt thereof-containing hydrophilic polymer comprises a lifitegrast or a salt thereof-containing hyaluronic acid polymer.

In non-limiting illustrative embodiments, as may be combined with one or more of the preceding paragraphs, the lifitegrast or a salt thereof-containing hydrophilic polymer comprises lifitegrast or a salt thereof attached to the hydrophilic polymer via the releasable linking group.

In non-limiting illustrative embodiments, as may be combined with one or more of the preceding paragraphs, the releasable linking group attached to the lifitegrast or a salt thereof and the hydrophilic polymer is one of a pH-labile bond, an acid-labile bond, a base-labile bond, an oxidatively labile bond, a metabolically labile bond, a biochemically labile bond, an enzyme-labile bond, a chemical-labile bond or a photo-labile bond.

In non-limiting illustrative embodiments, as may be combined with one or more of the preceding paragraphs, the lifitegrast or a salt thereof-containing hydrophilic polymer comprises a lifitegrast or a salt thereof-containing glycosaminoglycan polymer.

In non-limiting illustrative embodiments, as may be combined with one or more of the preceding paragraphs, the lifitegrast or a salt thereof-containing glycosaminoglycan polymer comprises a glycosaminoglycan having a polymer backbone and a first side chain comprising a lifitegrast or a salt thereof residue comprising the releasable linking group grafted onto the polymer backbone, and a second side chain comprising an ethylenically unsaturated reactive-containing residue grafted onto the polymer backbone.

In non-limiting illustrative embodiments, as may be combined with one or more of the preceding paragraphs, the ethylenically unsaturated reactive group comprises a (meth)acrylate group.

In non-limiting illustrative embodiments, as may be combined with one or more of the preceding paragraphs, the ophthalmic device-forming comonomer comprises one or more hydrophilic monomers.

In non-limiting illustrative embodiments, as may be combined with one or more of the preceding paragraphs, the one or more hydrophilic monomers are selected from the group consisting of an amide, cyclic lactam, hydroxyl-containing (meth)acrylate, poly(alkene glycols) functionalized with polymerizable groups and mixtures thereof.

In non-limiting illustrative embodiments, as may be combined with one or more of the preceding paragraphs, amide is selected from the group consisting of N,N-dimethylacrylamide, N,N-dimethylmethacrylamide and mixtures thereof.

In non-limiting illustrative embodiments, as may be combined with one or more of the preceding paragraphs, the cyclic lactam is selected from the group consisting of N-vinyl-2-pyrrolidone, N-vinyl caprolactam, N-vinyl-2-piperidone and mixtures thereof.

In non-limiting illustrative embodiments, as may be combined with one or more of the preceding paragraphs, the ophthalmic device-forming comonomer comprises one or more silicone monomers.

In non-limiting illustrative embodiments, as may be combined with one or more of the preceding paragraphs, the one or more silicone monomers comprise a non-bulky organosilicon-containing monomer.

In non-limiting illustrative embodiments, as may be combined with one or more of the preceding paragraphs, the non-bulky organosilicon-containing monomer is represented by the following structure:

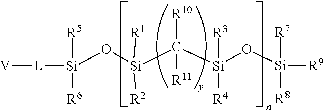

wherein V is ethylenically unsaturated polymerizable group, L is a linker group or a bond; R¹, R², R³, R⁴, R⁵, R⁶, R⁷, R⁸, and R⁹ are independently hydrogen an alkyl group, a haloalkyl group, a cycloalkyl group, a heterocycloalkyl group, an alkenyl group, a halo alkenyl group, or an aromatic group; R¹⁰ and R¹¹ are independently hydrogen or alkyl wherein at least one of R¹⁰ and R¹¹ is hydrogen; y is 2 to 7 and n is 1 to 100, or the non-bulky organosilicon-containing monomer is represented by the following structure:

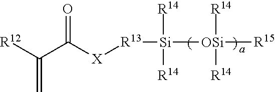

wherein R¹² is H or methyl; X is O or NR¹⁶; wherein R¹⁶ is hydrogen or C₁ to C₄ alkyl, which may be further substituted with one or more hydroxyl groups; R¹³ is a divalent alkyl group, which may further be functionalized with a group selected from the group consisting of an ether group, a hydroxyl group, a carbamate group and combinations thereof, each R¹⁴ is independently a phenyl or C₁ to C₄ alkyl which may be substituted with fluorine, hydroxyl or an ether; R¹⁵ is a C₁ to C₄ alkyl; and a is 2 to 50.

In non-limiting illustrative embodiments, as may be combined with one or more of the preceding paragraphs, the one or more silicone monomers comprise a bulky siloxane monomer.

In non-limiting illustrative embodiments, as may be combined with one or more of the preceding paragraphs, the bulky siloxane monomer is selected from the group consisting of a bulky polysiloxanylalkyl (meth)acrylic monomer, a bulky polysiloxanylalkyl carbamate monomer and mixtures thereof.

In non-limiting illustrative embodiments, as may be combined with one or more of the preceding paragraphs, the bulky siloxane monomer is represented by the following structure:

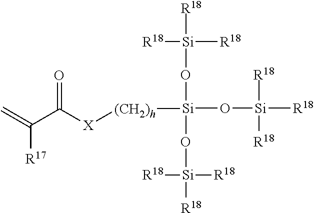

wherein X denotes —O— or —NR¹⁹— where each R¹⁹ is hydrogen or a C₁-C₄ alkyl; R¹⁷ independently denotes hydrogen or methyl; each R⁸ independently denotes a lower alkyl radical, a phenyl radical or a group represented by the following structure:

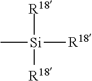

wherein each R^{18′} independently denotes a lower alkyl radical or a phenyl radical; and h is 1 to 10; or the following structure:

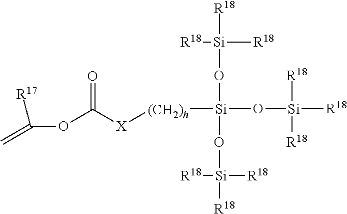

wherein X denotes —NR¹⁹—; wherein R¹⁹ denotes hydrogen or a C₁-C₄ alkyl; R¹⁷ denotes hydrogen or methyl; each R⁸ independently denotes a lower alkyl radical, a phenyl radical or a group represented by the following structure:

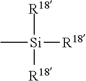

wherein each R^{18′} independently denotes a lower alkyl radical or a phenyl radical; and h is 1 to 10.

In non-limiting illustrative embodiments, as may be combined with one or more of the preceding paragraphs, the monomeric mixture further comprises an ultraviolet blocker.

In non-limiting illustrative embodiments, as may be combined with one or more of the preceding paragraphs, the monomeric mixture further comprises a blue light blocker.

In non-limiting illustrative embodiments, as may be combined with one or more of the preceding paragraphs, the lifitegrast or a salt thereof-containing hydrophilic polymer is present in the monomeric mixture in an amount ranging from about 0.1 wt. % to about 30 wt. %, based on the total weight of the monomeric mixture.

In non-limiting illustrative embodiments, as may be combined with one or more of the preceding paragraphs, the lifitegrast or a salt thereof-containing hydrophilic polymer is present in the monomeric mixture in an amount ranging from about 1 wt. % to about 10 wt. %, based on the total weight of the monomeric mixture.

In non-limiting illustrative embodiments, as may be combined with one or more of the preceding paragraphs, the ophthalmic device-forming comonomer is present in the monomeric mixture in an amount ranging from about 0.1 wt. % to about 98 wt. %, based on the total weight of the monomeric mixture.

In non-limiting illustrative embodiments, as may be combined with one or more of the preceding paragraphs, the ophthalmic device-forming comonomer is present in the monomeric mixture in an amount ranging from about 0.1 wt. % to about 90 wt. %, based on the total weight of the monomeric mixture.

In non-limiting illustrative embodiments, as may be combined with one or more of the preceding paragraphs, the ophthalmic device is a contact lens.

In non-limiting illustrative embodiments, as may be combined with one or more of the preceding paragraphs, the contact lens is a soft contact lens.

According to yet another aspect of the present disclosure, a method for delivering lifitegrast or a salt thereof to an eye of a subject, comprising:
(a) placing the ophthalmic device according to any one of the preceding paragraphs in an eye of a subject, wherein the ophthalmic device is a polymerization product of a monomeric mixture comprising (i) a lifitegrast or a salt thereof-containing hydrophilic polymer comprising a releasable linking group and an ethylenically unsaturated reactive group, and (ii) an ophthalmic device-forming comonomer to polymerization conditions to provide a polymerized contact lens, and
(b) delivering the lifitegrast or a salt thereof to an eye of a subject by contacting the contact lens with one of light having a wavelength of about 300 nanometers (nm) to about 700 nm or with an esterases or enzymes on the eye, in a tear film or on a flora of the eye.

In non-limiting illustrative embodiments, as may be combined with one or more of the preceding paragraphs, the lifitegrast or a salt thereof is delivered to the eye of the subject in a sustained release manner.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

1. An ophthalmic device comprising a polymerization product of a monomeric mixture comprising (a) a lifitegrast or a salt thereof-containing hydrophilic polymer comprising a releasable linking group and an ethylenically unsaturated reactive group; and (b) an ophthalmic device-forming comonomer.

2. The ophthalmic device according to claim 1, wherein the lifitegrast or a salt thereof-containing hydrophilic polymer comprises a lifitegrast or a salt thereof-containing glycosaminoglycan polymer.

3. The ophthalmic device according to claim 1, wherein the lifitegrast or a salt thereof-containing hydrophilic polymer comprises a lifitegrast or a salt thereof-containing hyaluronic acid polymer.

4. The ophthalmic device according to claim 1, wherein the lifitegrast or a salt thereof-containing hydrophilic polymer comprises lifitegrast or a salt thereof attached to the hydrophilic polymer via the releasable linking group.

5. The ophthalmic device according to claim 4, wherein the releasable linking group attached to the lifitegrast or a salt thereof and the hydrophilic polymer is one of a pH-labile bond, an acid-labile bond, a base-labile bond, an oxidatively labile bond, a metabolically labile bond, a biochemically labile bond, an enzyme-labile bond, a chemical-labile bond or a photo-labile bond.

6. The ophthalmic device according to claim 1, wherein the lifitegrast or a salt thereof-containing hydrophilic polymer comprises a glycosaminoglycan having a polymer backbone and a first side chain comprising lifitegrast or a salt thereof residue comprising the releasable linking group grafted onto the polymer backbone and a second side chain comprising an ethylenically unsaturated reactive-containing residue grafted onto the polymer backbone.

7. The ophthalmic device according to claim 1, wherein the ethylenically unsaturated reactive group comprises a (meth)acrylate group.

8. The ophthalmic device according to claim 1, wherein the ophthalmic device-forming comonomer comprises one or more hydrophilic monomers.

9. The ophthalmic device according to claim 1, wherein the ophthalmic device-forming comonomer comprises one or more silicone monomers.

10. The ophthalmic device according to claim 9, wherein the one or more silicone monomers comprise a non-bulky organosilicon-containing monomer.

11. The ophthalmic device according to claim 10, wherein the non-bulky organosilicon-containing monomer is represented by the following structure: 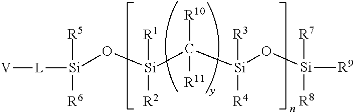
wherein V is ethylenically unsaturated polymerizable group, L is a linker group or a bond; R¹, R², R³, R⁴, R⁵, R⁶, R⁷, R⁸, and R⁹ are independently hydrogen an alkyl group, a haloalkyl group, a cycloalkyl group, a heterocycloalkyl group, an alkenyl group, a halo alkenyl group, or an aromatic group; R¹⁰ and R¹¹ are independently hydrogen or alkyl wherein at least one of R¹⁰ and R¹¹ is hydrogen; y is 2 to 7 and n is 1 to 100, or the non-bulky organosilicon-containing monomer is represented by the following structure: 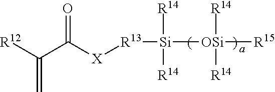
wherein R¹² is H or methyl; X is O or NR¹⁶; wherein R¹⁶ is hydrogen or C₁ to C₄ alkyl, which may be further substituted with one or more hydroxyl groups; R¹³ is a divalent alkyl group, which may further be functionalized with a group selected from the group consisting of an ether group, a hydroxyl group, a carbamate group and combinations thereof, each R¹⁴ is independently a phenyl or C₁ to C₄ alkyl which may be substituted with fluorine, hydroxyl or an ether; R¹⁵ is a C₁ to C₄ alkyl; and a is 2 to 50.

12. The ophthalmic device according to claim 9, wherein the one or more silicone monomers comprise a bulky siloxane monomer.

13. The ophthalmic device according to claim 12, wherein the bulky siloxane monomer is represented by the following structure: 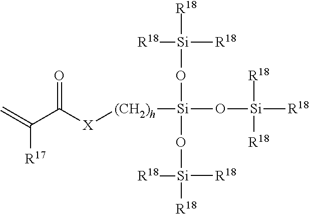
wherein X denotes —O— or —NR¹⁹— where each R¹⁹ is hydrogen or a C₁-C₄ alkyl; R¹⁷ independently denotes hydrogen or methyl; each R⁸ independently denotes a lower alkyl radical, a phenyl radical or a group represented by the following structure: 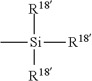
wherein each R^{18′} independently denotes a lower alkyl radical or a phenyl radical; and h is 1 to 10; or the following structure: 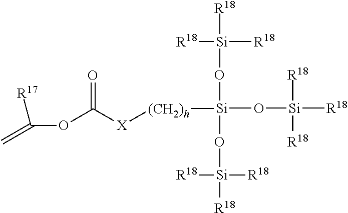
wherein X denotes —NR¹⁹—; wherein R¹⁹ denotes hydrogen or a C₁-C₄ alkyl; R¹⁷ denotes hydrogen or methyl; each R¹⁸ independently denotes a lower alkyl radical, a phenyl radical or a group represented by the following structure: 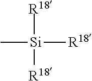
wherein each R^{18′} independently denotes a lower alkyl radical or a phenyl radical; and h is 1 to 10.

14. The ophthalmic device according to claim 1, wherein the monomeric mixture further comprises an ultraviolet blocker, a blue light blocker, or both.

15. The ophthalmic device according to claim 1, wherein the lifitegrast or a salt thereof-containing hydrophilic polymer is present in the monomeric mixture in an amount ranging from about 0.1 wt. % to about 30 wt. %, based on the total weight of the monomeric mixture.

16. The ophthalmic device according to claim 1, wherein the ophthalmic device is a contact lens.

17. The ophthalmic device according to claim 16, wherein the contact lens is a soft contact lens.

18. The ophthalmic device according to claim 1, wherein the lifitegrast or a salt thereof is released in a sustained release manner.

19. A method for making an ophthalmic device, comprising:
(a) subjecting a monomeric mixture comprising (i) a lifitegrast or a salt thereof-containing hydrophilic polymer comprising a releasable linking group and an ethylenically unsaturated reactive group, and (ii) an ophthalmic device-forming comonomer to polymerization conditions to provide a polymerized ophthalmic device; and
(b) hydrating the polymerized ophthalmic device.

20. (canceled)
